(19) United States
(12) Patent Application Publication
(10) Pub. No.: US 2018/0303532 A1
(43) Pub. Date: Oct. 25, 2018

(54) Delivery Device and Methods of Use

(71) Applicant: KT, LLC, Germantown, TN (US)

(72) Inventors: Timothy R. Brahm, Germantown, TN (US); Kevin Foley, Germantown, TN (US)

(21) Appl. No.: 16/021,577

(22) Filed: Jun. 28, 2018

Related U.S. Application Data
(63) Continuation-in-part of application No. 15/213,523, filed on Jul. 19, 2016, now Pat. No. 10,064,673.
Application No. 15/213,523 is a continuation of application No. 14/215,719, filed on Mar. 17, 2014, now Pat. No. 9,408,650.
(60) Provisional application No. 61/787,867, filed on Mar. 15, 2013.

(51) Int. Cl. A61B 17/88; A61L 27/38

(52) U.S. Cl. CPC A61B 17/8836; A61L 27/3847; A61B 2017/0023; A61L 2400/06; A61L 2430/02; A61B 17/8802

(57) ABSTRACT

A mixing and delivery device for flowable, homogenous material compositions and methods of use are provided. A kit for use by a medical professional is also provided.

### CROSS-REFERENCE TO RELATED APPLICATIONS

This application is a continuation-in-part of U.S. Ser. No. 15/213,523, filed Jul. 19, 2016, which is a continuation of U.S. Ser. No. 14/215,719, filed Mar. 17, 2014, now U.S. Pat. No. 9,408,650, which claims priority to U.S. Provisional Patent Application No. 61/787,867 filed Mar. 15, 2013, the contents of which are each incorporated herein in their entirety.

### FIELD OF THE INVENTION

The present invention is directed to a device for the controlled mixing and delivery of a flowable material composition to a site on or within the human body.

### BACKGROUND OF THE INVENTION

Flowable compositions are often used in medical and regenerative applications. Particularly, injectable bone substitute materials and various other flowable compositions are used for the repair and augmentation of orthopedic fractures and various other defects, including soft tissue defects. Bone substitute materials can also be used in certain diagnostic or therapeutic procedures that require the formation of a cavity in a bone mass. Bone substitute materials can be used to treat any bone, for example, bone which due to osteoporosis, avascular necrosis, cancer, or trauma, is fractured or is prone to compression fracture or collapse. These conditions, if not successfully treated, can result in deformities, chronic complications, and an overall adverse impact upon the quality of life.

### SUMMARY OF THE INVENTION

According to one aspect, a mixing and delivery device is provided. The mixing and delivery device includes a liquid chamber adapted to receive and mix a first liquid composition and a second liquid composition to form a flowable material composition. According to one embodiment, the flowable material composition is homogenous. The mixing and delivery device also includes a liquid delivery port located on a proximal end of the liquid chamber. The mixing and delivery device also includes a barrel, a plunger, and a suction port located on a distal end of the plunger. The device is adapted to deliver a controlled amount of flowable, homogenous material composition to a particular site. According to one embodiment, the plunger is adapted to engage the barrel. According to one embodiment,

the flowable, homogenous material composition hydrates or coats a site. According to one embodiment, the flowable, homogenous material composition flows at normal atmosphere, under positive pressure, or under negative pressure. According to one embodiment, the first and second liquid compositions are human birth tissue composition, carrier composition, patient-derived composition, or any combination thereof. According to one embodiment, the human birth tissue includes morselized amniotic membrane and amniotic fluid. According to one embodiment, the carrier composition includes Minimum Essential Medium, Dulbecco's Modified Eagle's Medium (DMEM), and human albumin 25% solution. According to one embodiment, the carrier composition includes Plasma Lyte-A which, in turn, includes sodium chloride, sodium gluconate, sodium acetate trihydrate, potassium chloride, and magnesium chloride. According to one embodiment, the suction port is adapted to receive a source of air pressure or vacuum. According to one embodiment, the device is disposable. According to one embodiment, the liquid chamber is in liquid communication with a liquid delivery port.

According to another aspect, a kit for use by a medical professional is provided. The kit includes at least one device as provided herein.

According to another aspect, a method of delivering a flowable, homogenous material composition is provided. The method includes the steps of drawing a first liquid composition and a second liquid composition into the liquid chamber of a device as provided herein to form a flowable, homogenous material composition and dispensing the flowable, homogenous material composition to a particular site to coat or hydrate the site. According to one embodiment, the method further includes the step of immersing the liquid delivery port located on a proximal end of the liquid chamber port in the first liquid composition and drawing the first liquid composition into the liquid chamber under negative pressure. According to one embodiment, the method further includes the step of immersing the liquid delivery port located on a proximal end of the liquid chamber port in the second liquid composition and drawing the second liquid composition into the liquid chamber under negative pressure. According to one embodiment, the first and second liquid compositions may include human birth tissue composition, carrier composition, patient-derived composition, or any combination thereof. According to one embodiment, the human birth tissue includes morselized amniotic membrane and amniotic fluid. According to one embodiment, the carrier composition includes Minimum Essential Medium, Dulbecco's Modified Eagle's Medium (DMEM), and human albumin 25% solution. According to one embodiment, the carrier composition includes Plasma Lyte-A which, in turn, includes sodium chloride, sodium gluconate, sodium acetate trihydrate, potassium chloride, and magnesium chloride. According to one embodiment, the flowable, homogenous material composition flows at normal atmosphere, under positive pressure, or under negative pressure.

### BRIEF DESCRIPTION OF THE DRAWINGS

FIG. 1 illustrates a perspective view of a device according to one embodiment.

FIG. 2 illustrates a side view of a bone substitute chamber according to one embodiment.

FIG. 3 illustrates a side view of a bone substitute chamber according to one embodiment.

FIG. 4 illustrates a gasket according to one embodiment.

FIG. 5 illustrates a perspective view of a device according to another embodiment.

### DETAILED DESCRIPTION OF THE INVENTION

The present disclosure will now be described more fully hereinafter with reference to exemplary embodiments thereof. These exemplary embodiments are described so that this disclosure will be thorough and complete, and will fully convey the scope of the disclosure to those skilled in the art. Indeed, the present disclosure may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise. As used in the specification, and in the appended claims, the words “optional” or “optionally” mean that the subsequently described event or circumstance can or cannot occur.

As used herein, the term “human birth tissue” includes, but is not limited to, elements of the placental organ such as, for example, the placental globe, umbilical cord, associated membranes (chorion, amnion and amniotic membrane), other gelatins, fluids, cells and extracellular material obtained from a seronegative, healthy human.

As used herein, the term “bone substitute material” refers to any commercially available natural or synthetic osseous tissue utilized in the repair and reconstruction of human bones (e.g., demineralized bone matrix (DBM)).

As used herein, a “patient-derived composition” refers to a composition that includes at least one biological component obtained or derived from the patient receiving treatment. In certain embodiments, the patient-derived composition includes blood, blood components (e.g., platelet-rich plasma) or a combination thereof that is obtained from the patient.

As used herein, the terms “material composition” and “mixture” may be used interchangeably and refers to the substance that is formed and dispensed from the mixing and delivery device described herein. The material composition may be homogenous as a result of mixing.

As used herein, the term “flowable” refers to the ability of a liquid composition to flow or be flowed.

A device adapted to mix a first and second liquid to form a flowable, homogenous material composition. The device is particularly suited to dispense the flowable, homogenous material composition to a particular site to coat or hydrate the site. According to one embodiment, the flowable, homogenous material composition flows at normal atmosphere, under positive pressure, or under negative pressure. The mixing and delivery device includes a liquid chamber adapted to receive and mix a first liquid composition and a second liquid composition to form a flowable, homogenous material composition. The mixing and delivery device also includes a liquid delivery port located on a proximal end of the liquid chamber. The mixing and delivery device also includes a barrel, a plunger, and a suction port located on a distal end of the plunger. The device is adapted to deliver a controlled amount of flowable, homogenous material composition to a particular site. According to one embodiment, the plunger is adapted to engage the barrel. According to one embodiment, the flowable, homogenous material composition hydrates or coats a site. According to one embodiment, the flowable, homogenous material composition flows at normal atmosphere, under positive pressure, or under negative pressure. According to one embodiment, the first and second liquid compositions are human birth tissue composition, carrier composition, patient-derived composition, or any combination thereof. According to one embodiment, the human birth tissue includes morselized amniotic membrane and amniotic fluid. According to one embodiment, the carrier composition includes Minimum Essential Medium, Dulbecco's Modified Eagle's Medium (DMEM), and human albumin 25% solution. According to one embodiment, the carrier composition includes Plasma Lyte-A which, in turn, includes sodium chloride, sodium gluconate, sodium acetate trihydrate, potassium chloride, and magnesium chloride. According to one embodiment, the suction port is adapted to receive a source of air pressure or vacuum. According to one embodiment, the device is disposable. According to one embodiment, the liquid chamber is in liquid communication with a liquid delivery port.

According to another aspect, a method of delivering a flowable, homogenous material composition is provided. The method includes the steps of drawing a first liquid composition and a second liquid composition into the liquid chamber of a device as provided herein to form a flowable, homogenous material composition and dispensing the flowable, homogenous material composition to a particular site to coat or hydrate the site. According to one embodiment, the method further includes the step of immersing the liquid delivery port located on a proximal end of the liquid chamber port in the first liquid composition and drawing the first liquid composition into the liquid chamber under negative pressure. According to one embodiment, the method further includes the step of immersing the liquid delivery port located on a proximal end of the liquid chamber port in the second liquid composition and drawing the second liquid composition into the liquid chamber under negative pressure. According to one embodiment, the first and second liquid compositions may include human birth tissue composition, carrier composition, patient-derived composition, or any combination thereof. According to one embodiment, the human birth tissue includes morselized amniotic membrane and amniotic fluid. According to one embodiment, the carrier composition includes Minimum Essential Medium, Dulbecco's Modified Eagle's Medium (DMEM), and human albumin 25% solution. According to one embodiment, the carrier composition includes Plasma Lyte-A which, in turn, includes sodium chloride, sodium gluconate, sodium acetate trihydrate, potassium chloride, and magnesium chloride. According to one embodiment, the flowable, homogenous material composition flows at normal atmosphere, under positive pressure, or under negative pressure.

According to a particular embodiment, the device as provided can mix at least one human birth tissue composition with at least one bone substitute material. The device is further particularly suited to allow a bone substitute material to optimally absorb a human birth tissue material composition thereby producing a single, homogenous material composition that can be delivered to a particular site on or within the human body. The device is further capable of being fully disposable.

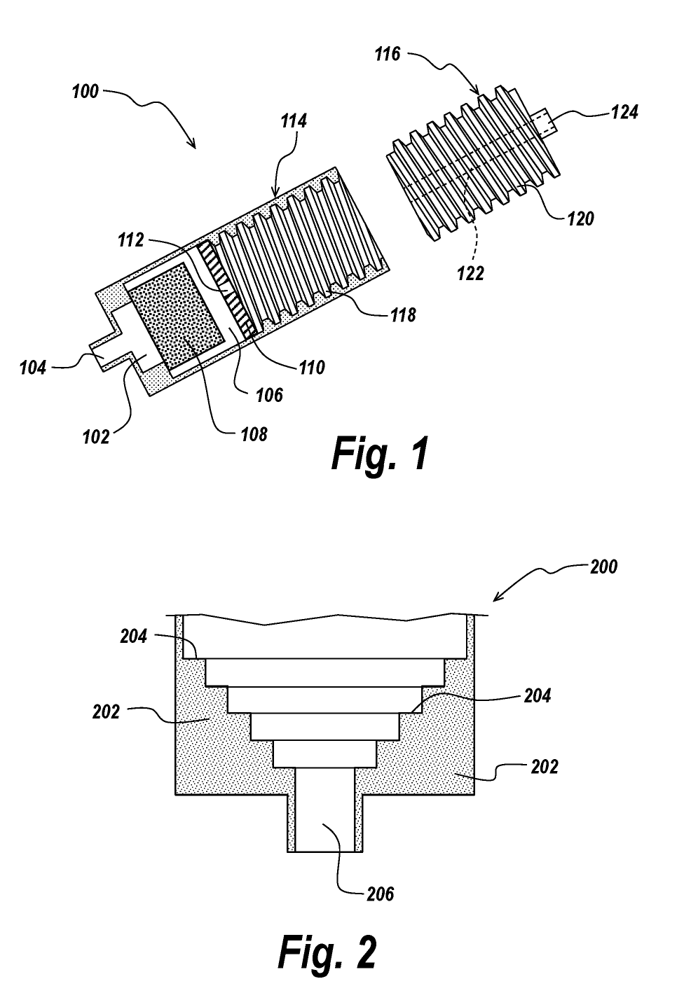

A particular embodiment of a mixing and delivery device is illustrated in FIG. 1. The device 100 includes a liquid chamber 102 that is in liquid connection with an optional liquid delivery port 104. A bone substitute chamber 106 is adapted to receive a bone substitute material 108. A gasket 110 is located in an upper portion of the bone substitute chamber 106. According to one embodiment, the gasket 110 includes at least one opening 112 (see FIG. 4) to allow for air flow from the bone substitute chamber and into the barrel 114. The barrel 114 includes internal threads 118 adapted to receive (i.e., complimentary to) external threads 120 located a plunger 116. The plunger 116 further includes a central channel or bore 122 that is aligned with the opening 112 of the gasket 110. The plunger 116 further includes a suction port 124 adapted to engage a source of negative air flow. The suction port 122 is substantially aligned with the central bore 118. According to an alternative embodiment, the gasket 110 may be attached to a proximal portion of the plunger 116.

FIG. 2 provides an alternative embodiment of the bone substitute chamber 200. As illustrated, the bone substitute chamber 200 includes support member 202 mounted to an internal surface of the bone substitute chamber 200. The support member 202 includes a plurality of steps 204 adapted to accommodate various sized bone substitute materials. Thus, the steps 204 allow a bone substitute material to center itself within the bone substitute chamber 200. The bone substitute chamber 200 may further include an optional liquid delivery port 206.

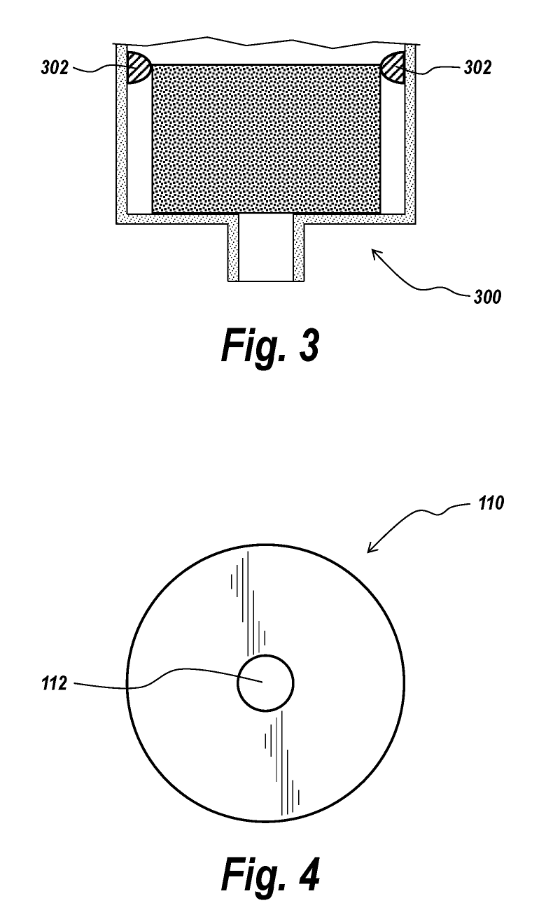

FIG. 3 provides another alternative embodiment of the bone substitute chamber 300. As illustrated, the bone substitute chamber 300 includes at least one seal 302 around an upper perimeter of the chamber 300. The seal 302 is adapted to secure a packed bone substitute material 304 within the chamber 300 prior to mixing.

According to one embodiment, each port (104, 124) includes the male portion of a Luer taper system. The Luer taper system is capable of receiving and securing a medical device having a corresponding female inlet or fitting. According to one embodiment, the Luer taper is a Luer-Lock™ design available from Becton Dickinson. According to another embodiment, the Luer taper system is a Luer-Slip™ design available from Becton Dickinson. Any Luer taper system commonly known in the art, however, may be used.

According to one embodiment, the liquid chamber of the device as described herein is capable of receiving at least one human birth tissue composition. According to one embodiment, the human birth tissue is processed such that the consistency and viscosity of the resulting human birth tissue composition is capable of being loaded and delivered via the device described herein. According to one embodiment, the human birth tissue is subject to moselization and homogenization prior to loading in the device. The human birth tissue composition may further include Minimum Essential Medium, Dulbecco's Modified Eagle's Medium (DMEM), Plasma Lyte-A, human albumin 25% solution, calcium-rich water, dimethyl sulfoxide, alkaline ionized water, and acidic ionized water.

According to one embodiment, the human birth tissue material composition may further include at least one carrier composition. The carrier composition may include Minimum Essential Medium, Dulbecco's Modified Eagle's Medium (DMEM), Plasma Lyte-A, human albumin 25% solution, calcium-rich water, alkaline ionized water, and acidic ionized water. The carrier may further include a variety of optional components to aid in disease resolution, healing, and recovery. Exemplary optional components include, but are not limited to, antibiotics, anti-inflammatory agents, anti-viral agents, growth factors, anti-proliferative agents, cytokines, antihistamines, pain medications, biocides, cellular attractant and scaffolding reagents (e.g., fibronectin), wound healing agents or sealants, nutritional agents (e.g., vitamins), hormones, alkylating agents, immunomodulatory agents (e.g., steroids), collagens, hyaluronic acid, waxes, glycols and derivatives thereof, glycercols and derivatives thereof, oils (including essential oils), fatty acids, cholesterols, alcohols, emollients, adsorbents, lubricants, emulsifying agents, thickening agents, humectants, surfactants, pharmaceutical ingredients, preservatives, antifungal agents, antioxidants, antimicrobial agents, structuring agents, dispersing agents, pH-adjusting components, sequestering or chelating agents, wetting agents, coloring agents, and/or other specialized proteins or small molecules known in the art to be suitable for use in a composition that can be applied onto or within the human body.

In use, the device may be initially loaded with a bone substitute material within the bone substitute chamber. According to one embodiment, the bone substitute material is loaded through the barrel. The bone substitute material may be a pre-packaged, single use bone substitute such as the Bonus™ II demineralized bone matrix product available from Biomet®. The plunger is then engaged or threaded within the barrel. If present, the liquid delivery port may then be immersed in a liquid composition (e.g., human birth tissue material composition or other suitable composition (e.g., saline)). Alternatively, in the absence of a liquid delivery port, the liquid chamber may be manually filled with the liquid composition either prior to or after loading the bone substitute material. The user may then attach or engage a source of negative pressure at the suction port. Once engaged, the negative pressure creates an air flow through the bore thereby drawing liquid composition up through the liquid chamber and into the bone substitute chamber. The negative pressure may be supplied by a vacuum pump or by via a vacuum syringe. By drawing up the liquid composition in this manner, the liquid fully infiltrates the bone substitute material allowing complete and optimal absorption of the liquid within the bone substitute material. In certain embodiments, the negative pressure may be adjusted such that a pre-determined amount of liquid is drawn into the bone substitute chamber and subsequently mixed with the bone substitute material. The resulting homogenous material may be removed by disengaging the plunger from the barrel and physically removing the homogenous bone substitute material. In another embodiment, the homogenous bone substitute material is dispensed back through the liquid delivery port by further advancing the plunger with the barrel. In yet another embodiment, the homogenous bone substitute material may be dispensed by reversing air flow (i.e., applying positive air flow) through the suction port such that the homogenous bone substitute material is forced out through the liquid delivery port.

The resulting homogenous bone substitute material may be in the form of a fine bead, flowable gel, or putty, depending on the amount of liquid drawn. If desired, the homogenous material may then be manipulated or shaped prior to placement on or within an osseous defect (e.g., bone void).

The device as described herein may be used to deliver a bone substitute within a variety of medical applications. In certain embodiments, the device may used in dental applications (e.g., periodontal or maxofacial surgery). In other embodiments, the device may be used in orthopedic applications for sealing, filling and/or otherwise treating bone voids within the body of a patient. In other embodiments, the device may be used for sealing, filling and/or otherwise treating a soft tissue defect in or on the body of a patient.

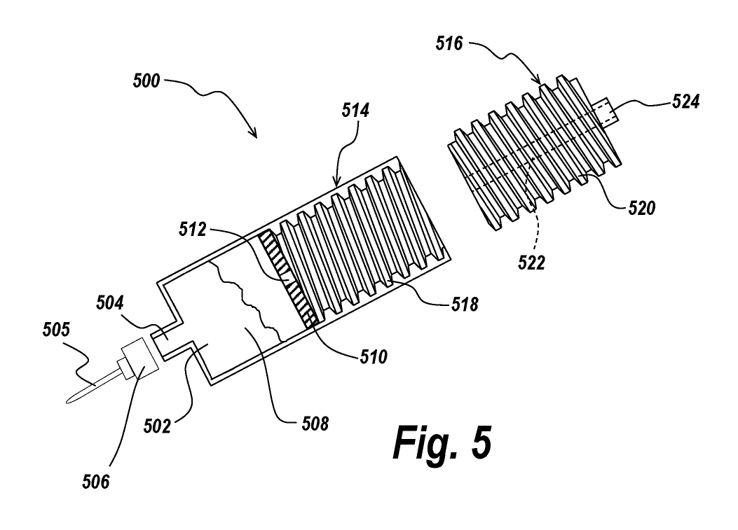

FIG. 5 provides an embodiment of a mixing and delivery device 500. The delivery device 500 includes a liquid chamber 502 that is in liquid connection with a liquid delivery port 504. According to a preferred embodiment, the liquid delivery port 504 includes the male portion of a Luer taper system. The Luer taper system is capable of receiving and securing a delivery member 505 having a corresponding female inlet or fitting 506. According to one embodiment, the Luer taper is a Luer-Lock™ design available from Becton Dickinson. According to another embodiment, the Luer taper system is a Luer-Slip™ design available from Becton Dickinson. However, any Luer taper system commonly known in the art may be used.

According to one embodiment, the delivery member 505 may be any component that provides an injection into or direct application over the chosen site. According to the illustrated embodiment of FIG. 5, the delivery member 505 is a needle. According to such an embodiment, modes of administration may include, but not be limited to: intramuscular, subcutaneous, intraperitoneal, percutaneous, soft tissue injection, intraosseous, intravenous, intravascular, intracerebral, transdermal, topical or mucosal. According to another embodiment, the delivery member 505 is a cannula or needle cannula.

The liquid chamber 502 receives and mixes at least two compositions and delivers a single flowable, homogenous material composition 508. A gasket 510 is located in an upper portion of the liquid chamber 502. According to one embodiment, the gasket 510 includes at least one opening 512 to allow for air flow from the liquid chamber 502 and into the barrel 514. The barrel 514 includes a plurality of internal threads 518 adapted to receive (i.e., complimentary to) external threads 520 located a plunger 516. The plunger 516 further includes a central channel or bore 522 that is aligned with the opening 512 of the gasket 510. The plunger 516 further includes a suction port 524 adapted to engage a source of air flow such as negative air flow. The suction port 524 is substantially aligned with the central bore 522. According to an alternative embodiment, the gasket 510 may be attached to a proximal portion of the plunger 516.

The delivery device of FIG. 5 is for adapted to mix at least a first liquid composition and a second liquid composition and deliver a single, flowable, homogenous material composition to a particular site on or within the human body. According to a particular embodiment, the liquid chamber receives and mixes two compositions and delivers a single homogenous material composition. According to a particular embodiment, the liquid chamber receives and mixes three compositions and delivers a single homogenous material composition. According to one embodiment, the flowable, homogenous material composition hydrates or coats a site. According to one embodiment, the flowable, homogenous material composition flows at normal atmosphere, under positive pressure, or under negative pressure.

According to one embodiment, the delivery device of FIG. 5 may mix at least one human birth tissue composition with at least one suitable carrier composition or a patient-derived composition. According to one embodiment, the delivery device of FIG. 5 may mix at least one human birth tissue composition with both a suitable carrier composition and at least one patient-derived composition. The delivery device is further capable of mixing any or all of the compositions and delivering the resulting flowable, homogenous material composition to a specific site on or within the human body. The delivery device is further capable of being fully disposable.

According to one embodiment, the liquid chamber of the delivery device of FIG. 5 is capable of receiving at least one human birth tissue composition. According to one embodiment, the human birth tissue includes morselized amniotic membrane and amniotic fluid. According to one embodiment, the human birth tissue includes morselized amnion and amniotic fluid. According to one embodiment, the human birth tissue includes morselized chorion and amniotic fluid. According to one embodiment, the human birth tissue is processed such that the consistency and viscosity of the resulting human birth tissue composition is capable of being loaded and delivered via the device described herein. According to one embodiment, the human birth tissue is subject to moselization and homogenization with amniotic fluid prior to loading in the device. The human birth tissue composition may further include Minimum Essential Medium, Dulbecco's Modified Eagle's Medium (DMEM), Plasma Lyte-A, human albumin 25% solution, calcium-rich water, dimethyl sulfoxide, alkaline ionized water, and acidic ionized water.

According to one embodiment, the liquid chamber of the delivery device of FIG. 5 is capable of receiving at least one carrier composition. The carrier composition may include one or more components such as, for example, Minimum Essential Medium, Dulbecco's Modified Eagle's Medium (DMEM), Plasma Lyte-A, human albumin 25% solution, calcium-rich water, alkaline ionized water, and acidic ionized water. The carrier may further include a variety of optional components to aid in disease resolution, healing, and recovery. Exemplary optional components include, but are not limited to, antibiotics, anti-inflammatory agents, anti-viral agents, growth factors, anti-proliferative agents, cytokines, antihistamines, pain medications, biocides, cellular attractant and scaffolding reagents (e.g., fibronectin), wound healing agents or sealants, nutritional agents (e.g., vitamins), hormones, alkylating agents, immunomodulatory agents (e.g., steroids), collagens, hyaluronic acid, waxes, glycols and derivatives thereof, glycercols and derivatives thereof, oils (including essential oils), fatty acids, cholesterols, alcohols, emollients, adsorbents, lubricants, emulsifying agents, thickening agents, humectants, surfactants, pharmaceutical ingredients, preservatives, antifungal agents, antioxidants, antimicrobial agents, structuring agents, dispersing agents, pH-adjusting components, sequestering or chelating agents, wetting agents, coloring agents, and/or other specialized proteins or small molecules known in the art to be suitable for use in a composition that can be applied onto or within the human body.

To use the device of FIG. 5, the plunger is engaged or threaded within the barrel. The liquid delivery port may then be immersed in a first liquid composition (e.g., human birth tissue material composition, carrier, patient-derived composition, or other suitable composition (e.g., saline)). The user may then attach or engage a source of negative pressure at the suction port. Once engaged, the negative pressure creates an air flow through the bore thereby drawing a first liquid composition up through the liquid delivery port and into the liquid chamber. The negative pressure may be supplied by a vacuum pump or by via a vacuum syringe. The liquid delivery port may then be immersed in a second liquid composition. Negative pressure is applied to the suction port to drawing up the second liquid composition (e.g., human birth tissue material composition, carrier, patient-derived composition, or other suitable composition (e.g., saline)). The aforementioned step may be optionally repeated for a third liquid composition (e.g., human birth tissue material composition, carrier, patient-derived composition, or other suitable composition (e.g., saline)). In certain embodiments, the negative pressure may be adjusted such that a pre-determined amount of liquid is drawn into the liquid chamber. The liquid compositions are then mixed in the liquid chamber. The resulting flowable, homogenous material composition (mixture) may be dispensed back through the liquid delivery port by further advancing the plunger with the barrel. In yet another embodiment, the homogenous material composition (mixture) may be dispensed by reversing air flow (i.e., applying positive air flow) through the suction port such that the homogenous material composition (mixture) is forced out through the liquid delivery port. According to one embodiment, the flowable, homogenous material composition hydrates or coats a site.

The device of FIG. 5 may be used to deliver a flowable, homogenous material composition (mixture) within a variety of medical applications. In certain embodiments, the device may be used in dental applications (e.g., periodontal or maxofacial surgery). In other embodiments, the device may be used in orthopedic applications for injecting the homogenous material composition within the body of a patient. In other embodiments, the device may be used for sealing, filling and/or otherwise treating a soft tissue defect in or on the body of a patient.

A kit for use by a medical professional is also provided. The kit includes at least one device as described herein. According to one embodiment, the liquid chamber may be pre-loaded with at least one, or a first, liquid composition (e.g., human birth tissue material composition, carrier, patient-derived composition, or other suitable composition (e.g., saline)). According to one embodiment, the kit is preserved as per applicable Food and Drug Administration guidelines. The kit further includes at least one set of instructions.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

We claim:

1. A mixing and delivery device comprising:
a liquid chamber adapted to receive and mix a first liquid composition and a second liquid composition to form a flowable, homogenous material composition;
a liquid delivery port located on a proximal end of the liquid chamber;
a barrel;
a plunger; and
a suction port located on a distal end of the plunger,
wherein the device is adapted to deliver a controlled amount of flowable, homogenous material composition to a particular site.

2. The device of claim 1, wherein the plunger is adapted to engage the barrel.

3. The device of claim 1, wherein the flowable, homogenous material composition hydrates or coats a site.

4. The device of claim 1, wherein the flowable, homogenous material composition flows at normal atmosphere, under positive pressure, or under negative pressure.

5. The device of claim 1, wherein the first and second liquid compositions are selected from the group consisting of human birth tissue composition, carrier composition, and patient-derived composition.

6. The device of claim 5, wherein the human birth tissue comprises morselized amniotic membrane and amniotic fluid.

7. The device of claim 5, wherein the carrier composition comprises Minimum Essential Medium, Dulbecco's Modified Eagle's Medium (DMEM), and human albumin 25% solution.

8. The device of claim 1, wherein the suction port is adapted to receive a source of air pressure or vacuum.

9. The device of claim 1, wherein the device is disposable.

10. The device of claim 1, wherein the liquid chamber is in liquid communication with a liquid delivery port.

11. A kit for use by a medical professional comprising:
a device according to claim 1.

12. A method of delivering a flowable, homogenous material composition comprising the steps of:
drawing a first liquid composition and a second liquid composition into the liquid chamber of the device of claim 1 to form a flowable, homogenous material composition; and
dispensing the flowable, homogenous material composition to a particular site to coat or hydrate the site.

13. The method of claim 12, further comprising the steps of immersing the liquid delivery port located on a proximal end of the liquid chamber port in the first liquid composition and drawing the first liquid composition into the liquid chamber under negative pressure.

14. The method of claim 13, further comprising the step of immersing the liquid delivery port located on a proximal end of the liquid chamber port in the second liquid composition and drawing the second liquid composition into the liquid chamber under negative pressure.

15. The method of claim 12, wherein the first and second liquid compositions are selected from the group consisting of human birth tissue composition, carrier composition, and patient-derived composition.

16. The method of claim 15, wherein the human birth tissue comprises morselized amniotic membrane and amniotic fluid.

17. The method of claim 15, wherein the carrier composition comprises Minimum Essential Medium, Dulbecco's Modified Eagle's Medium (DMEM), and human albumin 25% solution.

18. The method of claim 12, wherein the flowable, homogenous material composition flows at normal atmosphere, under positive pressure, or under negative pressure.
